(19) United States
(12) Patent Application Publication
(10) Pub. No.: US 2008/0183047 A1
(43) Pub. Date: Jul. 31, 2008

(54) System And Method For Identifying A Trigger Of An Illness

(76) Inventors: Anthony P. Geraci, New York, NY (US); Marc Grzeskowiak, Staten Island, NY (US)

Correspondence Address: WOLF, BLOCK, SHORR AND SOLIS-COHEN LLP, 250 PARK AVENUE, 10TH FLOOR, NEW YORK, NY, 10177, US

(21) Appl. No.: 11/668,774

(22) Filed: Jan. 30, 2007

(51) Int. Cl. A61B 5/00

(52) U.S. Cl. 600/300

(57) ABSTRACT

A system and method for identifying a potential trigger of an illness. Subject data including information about a subject, food consumption data including information about food consumed by the subject, and illness data including information about an illness experienced by the subject are forwarded to a server. The server also retrieves reference data indicating nutrient information for particular foods. The server determines the nutrients in each consumed food, determines various relative percentage weights for nutrients for times when an illness has been experienced, and outputs a report correlating experienced illness with consumed nutrients/foods. The server can also issue a report, based on the illness data, indicating whether particular medications are helpful in reducing the severity of illness. A report may also be generated to identify medication efficacy.

### BACKGROUND OF THE INVENTION

Many illnesses have unknown causes. For example, the direct cause of a migraine headache, an excruciating and sometime debilitating illness, is frequently unknown. However the headache may be attributable to some controllable stimulus like a particular food or medication. Some foods are known to cause migraines such as nuts or chocolate. However, analyzing a patient's food consumption and determining the appropriate offending stimulus is time consuming, arduous and prone to errors.

Prior art systems exist for detecting causes of some types of illnesses. For example, the system in U.S. Pat. No. 6,712,763 purports to look to certain patient activities and/or external stimuli (such as pollen count) to determine what may have caused a particular illness. Similarly, the system in U.S. Pat. No. 6,571,172 inquires about a user's intake of food and medication and then determines factors which may affect the health of the user over a period of time.

Other prior art systems request that a patient use a diary and enter all food consumed. The diary is then reviewed and analyzed for possible triggers of illness. It is difficult for a patient to maintain such a diary and difficult for a doctor to review. Moreover, the time of the day that each food is ingested may play a role in the onset of an illness yet such time data is frequently not tracked. Further, the amount of each food consumed may be a factor and even the nutritional make-up of each food. None of this data is tracked and analyzed in prior art systems.

Thus, there is a need in the art for a system which can correlate food consumption with illness and can identify potential food triggers that a patient should avoid.

### BRIEF DESCRIPTION OF THE DRAWINGS

FIG. 1 is a flow diagram illustrating a process in accordance with an embodiment of the invention.

FIG. 2 is a system diagram in accordance with an embodiment of the invention.

FIG. 3 is a chart illustrating a possible break down of food elements into food components in accordance with an embodiment of the invention.

FIG. 4 is a flow diagram illustrating a process in accordance with an embodiment of the invention.

FIG. 5 is a flow diagram illustrating a process in accordance with an embodiment of the invention.

FIG. 6 is a flow diagram illustrating a process in accordance with an embodiment of the invention.

### SUMMARY OF THE INVENTION

One embodiment of the invention is a method for identifying a potential trigger of an illness. The method comprises receiving food consumption data from a subject, the food consumption data including information relating to food consumed by the subject and receiving illness data from the subject, the illness data identifying an illness experienced by the subject. The method further comprises identifying a potential trigger for the illness based on the food consumption data.

Another embodiment of the invention is a system for identifying a potential trigger of an illness. The system comprises a first server effective to receive food consumption data from a subject, the food consumption data including information relating to food consumed by the subject, the first server further effective to receive illness data from the subject, the illness data identifying an illness experienced by the subject. The system further comprises a database, in communication with the first server, the database effective to store the food consumption data and the illness data; and a second server in communication with the database, the second server effective to identify a potential trigger for the illness based on the food consumption data.

### DETAILED DESCRIPTION OF THE PREFERRED EMBODIMENT(S)

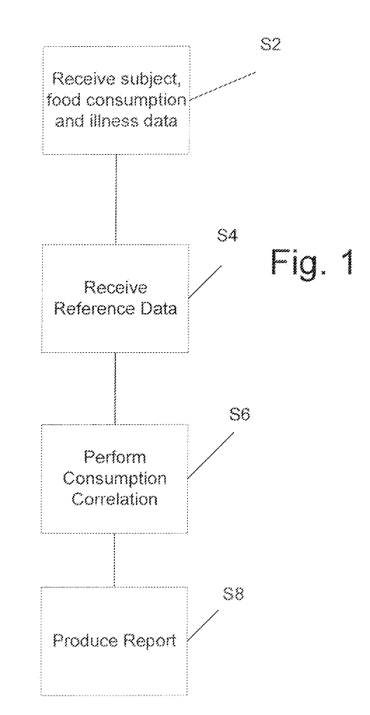

An example of a process which could be performed in accordance with an embodiment of the invention is shown in FIG. 1. At step S2, subject, food consumption, and illness data are received from a subject. Such subject and food consumption data may include information about a subject such as his height and weight and food and/or medicines consumed by subject along with associated times. Illness data may indicate an illness experienced by the subject and associated times. At step S4, reference data is received. The reference data may include data that does not change regularly such as publicly available data regarding food and its constituent nutrients. At step S6, a consumption correlation process is performed which determines relationships between food consumed by the subject and illness experienced by the subject. At step S8, a report is produced from the consumption correlation process.

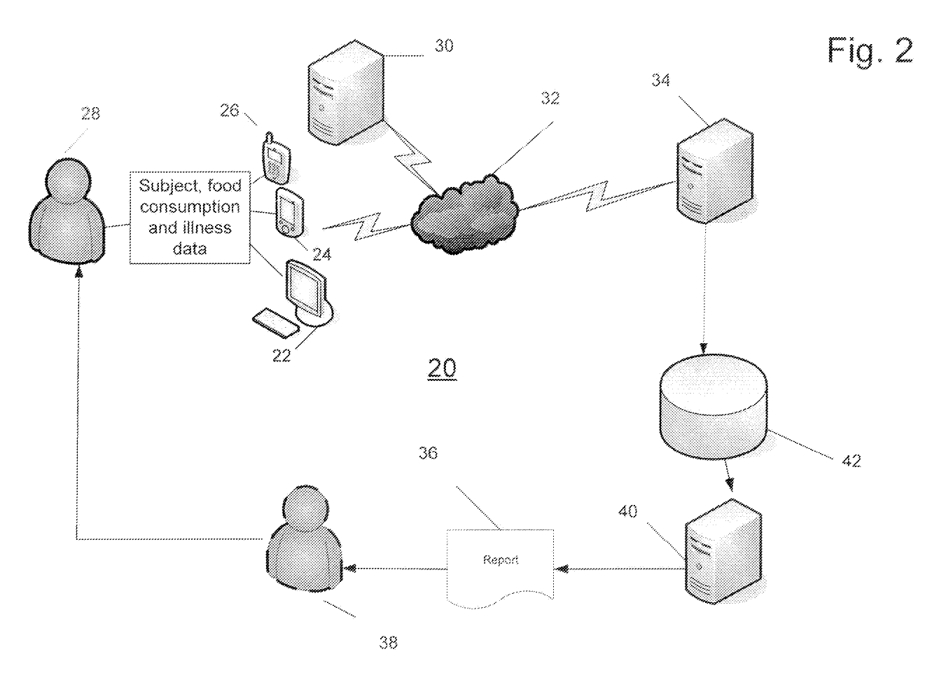

Referring to FIG. 2, there is shown a system 20 in accordance with an embodiment of the invention. System 20 includes a data collection server 34 effective to capture data from a plurality of sources. Data collection server 34 may receive subject, food consumption, and illness data from a subject 28 through a variety of communication devices including, for example, a cell phone 26, a smart phone 24 or a computer 22. Subject 28 may communicate the subject, food consumption, and illness data through an applicable network 32, such as the Internet or a cellular network, to data collection server 34. For example, subject 28 may communicate with data collection server 34 via a secure web-based application using a standard web browser such as—INTERNET EXPLORER, NETSCAPE NAVIGATOR or FIREFOX. Other optional methods for communication between subject 28 and data collection server 34 include the use of smart keys and/or flash memory sticks with built-in barcode scanners.

The subject data sent by subject 28 includes attributes of subject 28 including, for example, personal information such as gender, ethnicity, menstrual cycle, address information, physical makeup such as height and weight, and relevant family and medical history. This data may be used in aggregate analysis and trending across subjects through data mining as is discussed in more detail below. For example, a portion of a produced database may be sold without patient names. Food consumption data may include a daily log of food consumed including a time when each food was consumed. Food includes, for example, anything that has entered a patient's body including any food, food substance, liquid, herbs, medication, supplement, or any other element ingested orally or manually injected. Food includes items that may be taken on their own, such as salt, and items made of multiple ingredients, such as a cheese burger which is made of multiple ingredients including salt.

Medication includes prescription and over-the-counter drugs as well as holistic substances. As discussed below, if possible, such holistic substances will broken down to their nutrient/chemical composition with consultation with, for example, a pharmaceutical database. If such information is not available, system 20 will obtain the most granular data available. Illness data includes information about illnesses experienced by the subject including, for example, a paroxysmal illness like a migraine. Illness data also includes a date and time when the illness was experienced.

Subject, food consumption, and illness data may be forwarded to data collection server 34 over periods when an illness is experienced and when an illness is not experienced. Data collection server 34 communicates subject, food consumption, and illness data to a data collection database 42. Database 42 stores all the data in a table associating each piece of data with a related time. An example of a partial table in database 42 is shown below.

| | |
|---|---|
| Bun | 2:00 PM |
| Hamburger | 2:00 PM |
| Pickle | 2:00 PM |
| Ketchup | 2:00 PM |
| Headache | 6:00 PM |

As an example describing the communication of data from subject 28, when subject 28 is using a browser and computer 22, data collection server 34 may provide a web page including a drop-down menu that may be used to input daily foods and medications for a defined period—for example, one, two or three months. Subject 28 may enter into the web page the time that each food was consumed. For the illness data, any illnesses, such as, for example, a migraine, may be entered along with the date and time that the subject experienced the illness; a severity—for example on a pain scale of 1-10; and a time when the subject stopped experiencing the illness. As part of the illness data, subject 28 may also communicate to data collection server 34 any treatment medications taken with each illness for treatment of the illness and a severity of the illness after, for example 1 hour, 2 hours and 24 hours. Examples of treatment medications include daily headache medications, abortive medications, etc. An example of a partial table which may be produced in database 42 as a result of these inputs is shown below.

| | |
|---|---|
| Headache Severity 7 | 6:00 PM |
| Abortive Medication X | 7:30 PM |
| Headache Severity 2 | 9:00 PM |
| No Headache | 10:30 PM |

As for food consumed, the web page provided by data collection server may display a menu including fields such as: breakfast, lunch, snacks and dinner. The subject may also be given choices for food categories such as: dairy products, breads, cereals, fruits, vegetables, pasta, meats, nuts, condiments, alcohol, non-alcoholic beverages, spices, processed foods, sweets, miscellaneous. These categories assist the subject in listing the food consumed.

Data collection server 34 further receives static reference data including, for example, reusable data elements that do not frequently change. Such static reference data may include a master food list, a master list of nutrients, a correlation between the master food list and the nutrient list, and referenced medications. The referenced medications may include a list of known medications gathered from either a centralized source or generated by system 20. The reference data may be compiled outside of system 20 on, for example, a server 30 and may be communicated to collection server 34 through, for example, network 32. For example, the master food and nutrient data may be obtained from the United States Department of Agriculture. Such data may be currently obtained on the World Wide Web at nal.usda.gov/fnic/foodcomp/bulletins.faq.html#copyright. Static reference data may be further supplemented from other data sources such as, for example, food and beverage manufacturers, the FDA and pharmaceutical companies. For each food, data collection server 34 will attempt to obtain information about the food down to the a molecular level. Data collection server 34 forwards the static reference data to database 42.

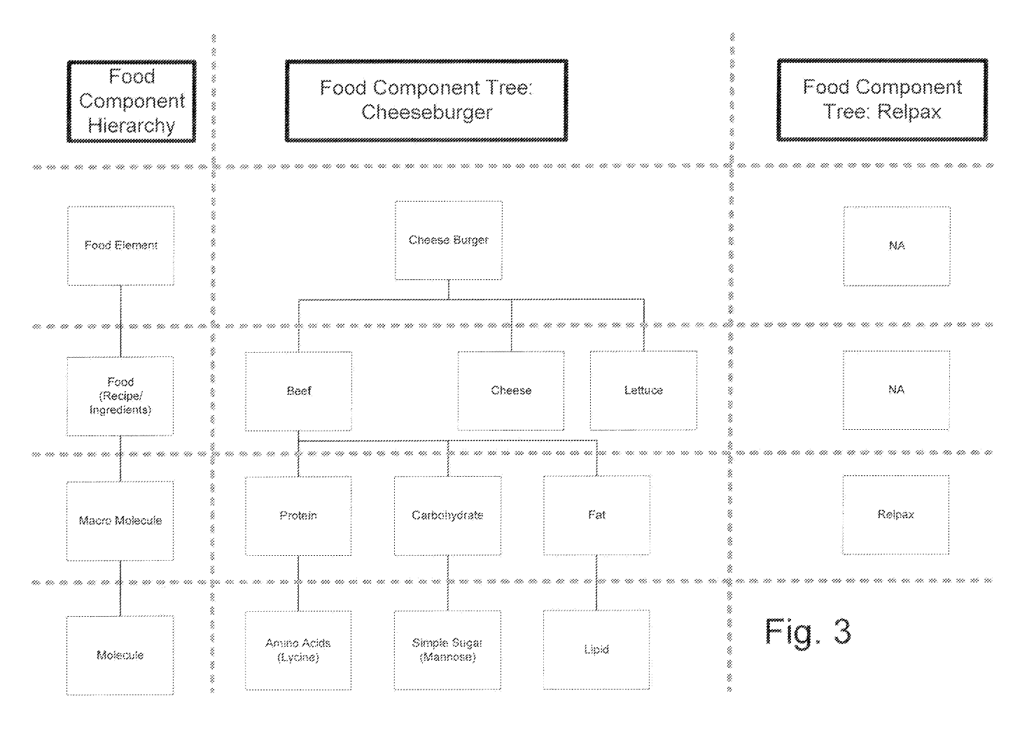

As shown at the table in FIG. 3, foods may be thought of a being assignable to one of four levels in a food component hierarchy. Clearly, the number four (4) is arbitrary and any number may be used. At a highest level in the hierarchy is a “food element” such as a “cheese burger”. Patients are given a convenient method of recording their food intake (e.g. “cheese burger”) and do not necessarily need to have knowledge of the underlying makeup of the food element. System 20 uses available databases to break down the food element into the lowest components possible. As shown in FIG. 3, if possible, the food element will be broken down into food ingredients or recipe—in the example beef, cheese and lettuce. Those ingredients may be broken down into macro molecules—such as protein, carbohydrate, and fat. Finally, the macro molecules may be broken down further into molecules—such as amino acids, simple sugars and lipids. Some foods may only fall into one level of the hierarchy—such as Relpax shown in the figure. In this way, any food consumed by a patient may be broken down into a tree having a plurality of levels. If only the food element is known, such as for example “grandma's meatloaf”, analysis will be performed on that level.

A consumption correlation server 40 receives by communication data stored in data collection database 42. Consumption correlation server 40 performs an analysis on the subject data, food consumption data, illness data and static reference data and produces a report 36 identifying potential triggers for the subject's illness. Report 36 may also illustrate the effects that certain medications had on the illness. Report 36 is optionally forwarded to a physician 38 for review and then to subject 28. Subject 28 may review report 36, modify his behavior and continue to forward subject, food consumption, and illness data to data collection server 34 so that the efficacy of treatment medications and/or changes in the patient's diet may be monitored for triggers of an illness.

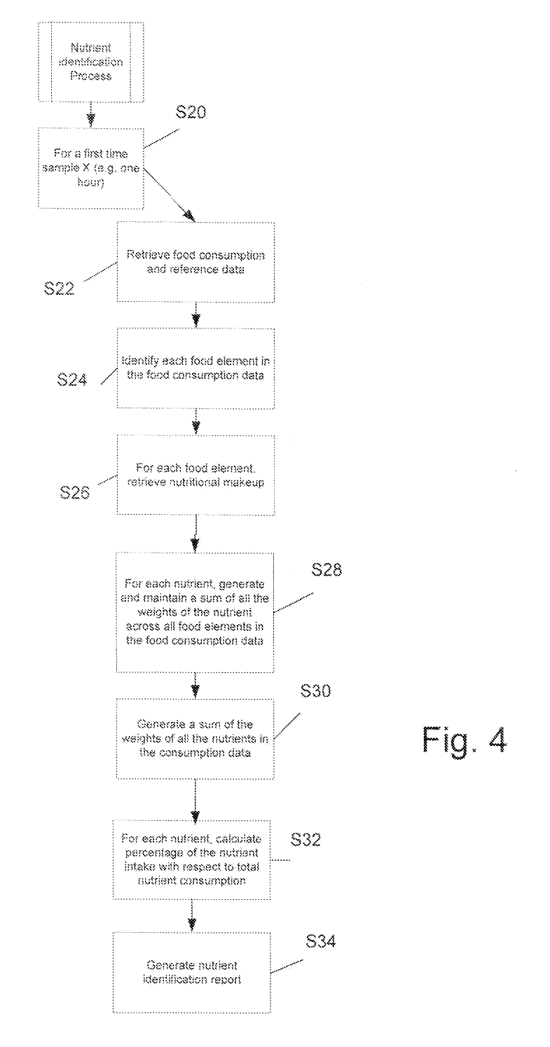

Consumption correlation server 40 performs at least three different types of processes on data from data collection database 42—a nutrient identification process, a correlation process and a treatment medication efficacy process. Referring to FIG. 4, there is shown an example of the nutrient identification process in accordance with an embodiment of the invention. The process shown could be performed in conjunction with, for example, system 20 of FIG. 2. At step S20, a loop is defined so that the following steps are repeatedly performed within a first time sample X.

For example, the loop may be performed for every hour of received data. At step S22, food consumption data and reference data are retrieved such as, for example, from database 34. At step S24, each food element in the food consumption data is identified.

At step S26, for each food element identified in the food consumption data, the nutritional makeup is retrieved listing the nutrients in the food consumed down to as close to the molecular level as is available. This lowest available level is called a nutrient and may be at the “food element” level of FIG. 3—such as in cases where the ingredients of the food element are not known. For example, the nutrient may be “lipid” or “grandma's meatloaf”. At step S28, for each particular nutrient, a running sum is generated corresponding to the weight of the particular nutrient across all food elements in the food consumption data during the first time sample. For example, 25 grams of sugar in the past hour. At step S30, a running sum is generated of all of the weights of all the nutrients in the food consumption data during the first time sample. At step S32, for each particular nutrient, a percentage is calculated of the weight of the particular nutrient consumed with respect to the weight of the total nutrient consumption in the first time sample. At step S34, a nutrient identification report is generated including the percentage weight of each nutrient, the total weight of each nutrient, and a list of all nutrients consumed during the first time sample.

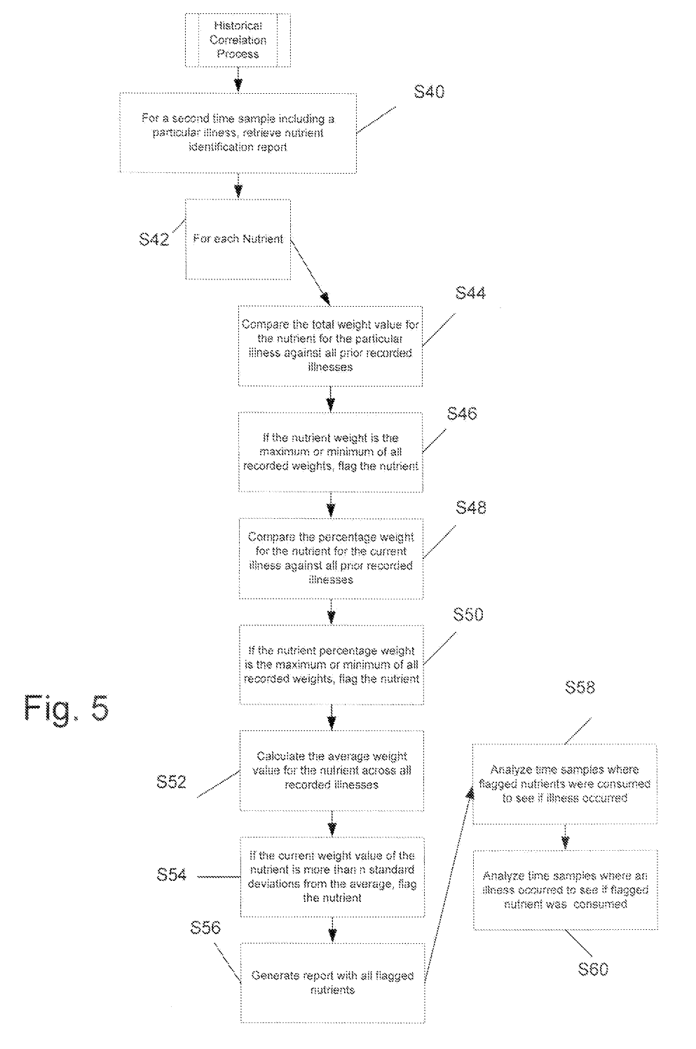

Referring to FIG. 5, there is shown an example of a historical correlation process in accordance with an embodiment of the invention. The historical correlation process could be implemented using, for example, system 20 of FIG. 2 and could be used in conjunction with the nutrient identification process of FIG. 4. At step S40, for a second time sample, at least one nutrient identification report is retrieved. The second time sample may be different from, and perhaps longer than, the first time sample discussed with reference to FIG. 4. The second time sample could be, for example, a period including an instance where the patient experienced a particular illness. For example, if a subject experienced an illness at time T, the historical correlation process may focus on a period 1 hour prior to time T. If nothing significant is identified during the time period, such as no food being consumed, the historical correlation process may focus on a period further back in time—such as three hours prior to time T. The nutrient identification report may indicate all nutrients consumed by the subject and the relative weight percentages of each nutrient compared with the total weights of all nutrients consumed by the subject during a first time sample.

At step S42, a loop is defined so that the following steps are performed for each particular nutrient. At step S44, a comparison is made of the weight of a particular nutrient during the second time sample including an illness against all prior recorded second time samples including other illnesses. At step S46, if the nutrient weight is the maximum or minimum weight of all recorded weights among all recorded time samples, then the nutrient is flagged. At step S48, the percentage weight of the nutrient compared with the weights of all nutrients consumed during the second time sample is compared against percentages in all prior recorded second time samples. At step S50, if the percentage weight of the nutrient is the maximum or minimum of all recorded weights, the nutrient is flagged.

At step S52, a calculation is made for the average weight value for the nutrient across all recorded values for all illnesses. At step S54, if the current weight value of the nutrient in the second time sample is n standard deviations from the average, (where n>1) the nutrient is flagged. At step S56, a report is generated with all flagged values. The flagged nutrients may also be combined to yield information higher up in the food hierarchy. For example, the flagged nutrient may be simple “grandma's meatloaf”. However, the flagged nutrients may also be “protein”, “carbohydrate” and “fat”. A comparison may be made between flagged nutrients and foods reported as being consumed by the subject during the second time sample so that these flagged nutrients may be combined into, for example, “cheese burger”. The comparison may, for example, refer to the food consumption data. The report could be sent to a doctor and the subject as is shown with report 36 in FIG. 2.

Once nutrients are identified as flagged, it is desirable to analyze: 1) time samples when the nutrient was consumed to see if the illness occurred S58 and 2) a time sample when an illness did occur to see if the same nutrient was consumed S60. For example, for each of the flagged nutrients, the system may look backward W number of days to see if an illness was experienced within T hours of consuming the nutrient. Similarly, the system may look at another illness period to see if any of the flagged nutrients were consumed. Any statistical technique known in the art may be used to combine these pieces of information. As is evident, as more time passes and more data is collected on experienced illnesses, data regarding potential triggers of such illnesses becomes more accurate and useful.

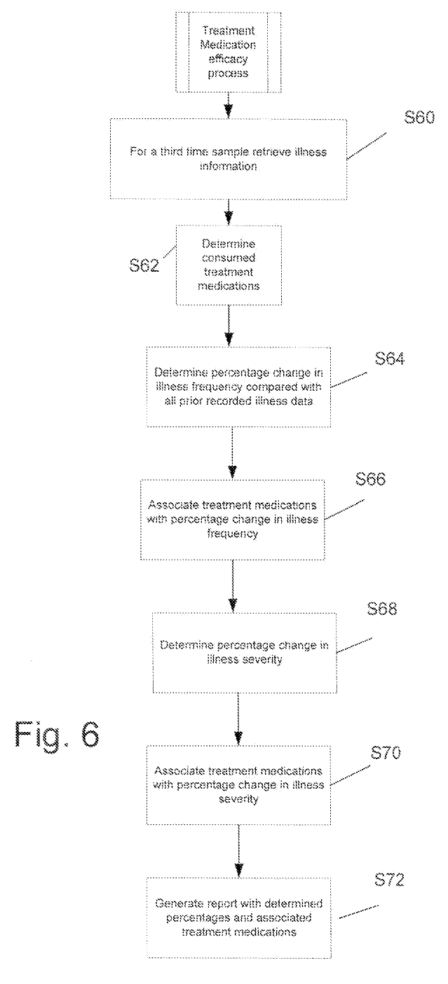

Referring to FIG. 6, there is shown a process which could be used in accordance with an embodiment of the invention. For example, the process of FIG. 6 could be used in conjunction with the system of FIG. 2 and/or the processes described with reference to FIG. 4 and FIG. 5. As shown in step S60, for a third time sample, illness information is retrieved. Such illness information could have been provided by a subject as was shown and described with reference to FIG. 2. For the retrieved illness information, in step S62, consumed treatment medications are determined. At step S64, the process determines, for the third time sample, a percentage change in illness frequency compared with all prior recorded illness data. At step S66, the treatment medications are associated with the percentage change in illness frequency. At S68, a determination is made as to the percentage change in illness severity in the third sample compared with prior recorded time samples. At S70, the treatment medications are associated with the percentage change in illness severity. At S70, a report is generated with the determined percentage changes and associated treatment medications. Clearly other reports can be generated and more examples of possible reports are discussed below.

Referring again to FIG. 2, consumption correlation server 40 includes processes and algorithms that effectively correlate food intake with the onset of an illness, such as a migraine headache, and identifies the effectiveness of associated medications. The algorithms performed by server 40 analyze food consumption trends in the period leading up to the illness and identify triggering candidates. The analyses goes even beyond high level food identification by also determining the nutrient makeup of foods.

For example, report 36 could indicate that eating pickles has an 80% chance of causing a migraine within 6 hours. Moreover, the system can determine the effectiveness of users' medications by determining a percent drop or increase in illness severity and frequency after consuming these medications.

Some forms of report 36 could include: the probability of having a headache within pre-defined intervals of ingesting any particular food or nutrient; the effect of mitigating factors such as migraine medications and menstrual cycle. For example, during time samples when an illness occurred, the system may identify a nutrient and a particular time in the menstrual cycle or a particular stress level. Any other life category may be tracked and may appear in the report. Other forms of report 36 could indicate: X foods have a high probability of causing the illness and should be avoided. Y foods should be monitored. Every time Z food was consumed, there was a 50% chance of an illness within 4 hours.

Other potential forms of report 36 could include: a food diary for individuals suspect of having food allergies; a tracking system for diabetic's food intake and insulin requirements; a research tool for groups seeking to correlate medication compliance with physiological parameters; a food diary for children on a ketone diet for control of seizures.

While preferred embodiments of the invention have described, the invention is not limited to those descriptions and other improvements and variations are known by those with ordinary skill in the art.

What is claimed is:

1. A method for identifying a potential trigger of an illness, the method comprising:
receiving food consumption data from a subject, the food consumption data including information relating to food consumed by the subject;
receiving illness data from the subject, the illness data identifying an illness experienced by the subject; and
identifying a potential trigger for the illness based on the food consumption data.

2. The method as recited in claim 1, wherein the food includes a medicine consumed by the subject.

3. The method as recited in claim 1, wherein the illness is a migraine.

4. The method as recited in claim 1, wherein the trigger is a food.

5. The method as recited in claim 1, wherein the report indicates a probability that the food/food element triggered the illness.

6. The method as recited in claim 1, further comprising:
receiving subject data from the subject, the subject data including attribute information about the subject; and
wherein the identifying includes identifying based on the subject data.

7. The method as recited in claim 1, further comprising:
receiving reference data; and
wherein the identifying includes identifying based on the reference data.

8. The method as recited in claim 1, further comprising identifying at least one nutrient in the food; and
wherein the identifying the potential trigger includes identifying the potential trigger based on the at least one nutrient.

9. The method as recited in claim 8, wherein the identifying at least one nutrient further comprises:
identifying a weight of the nutrient; and
calculating a percentage of the weight of the nutrient compared with a weight of the food.

10. The method as recited in claim 9, wherein the identifying the weight of the nutrient and the calculating the percentage are performed for a first time sample.

11. The method as recited in claim 10, further comprising:
flagging, for a second time sample including a particular illness, the nutrient when the nutrient weight of the nutrient is a minimum or maximum value of recorded nutrient weights in recorded second time samples including other illnesses; and
wherein the identifying the potential trigger is based on the flagging.

12. The method as recited in claim 10, further comprising:
flagging, for a second time sample including a particular illness, the nutrient when the weight percentage, calculated as the weight of the nutrient compared with the weight of the food, is a minimum or maximum value of recorded weight percentages in recorded second time samples including other illnesses; and
wherein the identifying the potential trigger is based on the flagging.

13. The method as recited in claim 10, further comprising:
flagging, for a second time sample including a particular illness, the nutrient when the weight of the nutrient is at least one standard deviation away from an average weight for the nutrient in recorded second time samples including other illnesses; and
wherein the identifying the potential trigger is based on the flagging.

14. The method as recited in claim 11, wherein the flagging is first flagging and further comprising:
second flagging, for the second time sample, the nutrient when the weight percentage, calculated as the weight of the nutrient compared with the weight of the food, is a minimum or maximum value of recorded weight percentages in the recorded second time samples including the other illnesses; and
wherein the identifying the potential trigger is based on the second flagging.

15. The method as recited in claim 14, further comprising:
third flagging, for the second time sample, the nutrient when the weight of the nutrient is at least one standard deviation away from an average weight for the nutrient in the recorded second time samples; and
wherein the identifying the potential trigger is based on the third flagging.

16. The method recited in claim 15, further comprising analyzing a fourth time sample where an illness was experienced to determine if one of the flagged nutrients was consumed during the fourth time sample.

17. The method as recited in claim 15, further comprising analyzing a fourth time sample when at least one of the flagged nutrients was consumed to see if the illness was experienced by the subject during the fourth time sample.

18. The method as recited in claim 1, wherein the illness information includes:
a treatment medication consumed by the subject at a first time; and
a second time when the patient experienced a decrease in a severity of the illness.

19. The method as recited in claim 16, further comprising:
determining, for a first time sample, a percentage change in illness frequency compared with recorded illness data;
associating the treatment medication with the percentage change; and generating a report including the percentage change and the treatment medication.

20. The method as recited in claim 16, further comprising:
determining, for a first time period, a percentage change in illness severity compared with recorded illness data;
associating the treatment medication with the percentage change; and
generating a report including the percentage change and the treatment medication.

21. The method as recited in claim 1, wherein the identifying includes generating a report.

22. A system for identifying a potential trigger of an illness, the system comprising:
a first server effective to receive food consumption data from a subject, the food consumption data including information relating to food consumed by the subject;
the first server further effective to receive illness data from the subject, the illness data identifying an illness experienced by the subject;
a database, in communication with the first server, the database effective to store the food consumption data and the illness data; and
a second server in communication with the database, the second server effective to identify a potential trigger for the illness based on the food consumption data.
